(19) United States
(12) Patent Application Publication
(10) Pub. No.: US 2026/0060844 A1
(43) Pub. Date: Mar. 5, 2026

(54) EYEWEAR ATTACHMENT ASSEMBLY CONNECTABLE TO HEADGEAR

(71) Applicant: Gauss Technologies LLC, Stamford, CT (US)

(72) Inventors: Stephen Joseph MASSIMI, Stamford, CT (US); Donald Scott PEEBLES, Bradenton, FL (US); Scott BEMIS, Prather, CA (US)

(21) Appl. No.: 18/823,963

(22) Filed: Sep. 4, 2024

(51) Int. Cl. A61F 9/02; A42B 1/247

(52) U.S. Cl. CPC A61F 9/025; A42B 1/247

(57) ABSTRACT

An eyewear attachment assembly is provided for configuring headgear to magnetically retain eyewear. The eyewear attachment assembly includes first and second attachment brackets, wherein a rearward side of each of the first and second attachment brackets includes a concave region configured for snap-connection onto a respective headgear feature, such as a pivot pin or a pivot bracket pivotably mounted on the pivot pin or a yoke of the headgear.

### TECHNICAL FIELD

This application relates generally to headgear and eyewear and, more specifically, to an eyewear attachment assembly adapted for retrofit connection to existing headgear such as ear protection headgear.

### BACKGROUND OF THE INVENTION

In certain industrial professions and sporting activities, it is desirable to wear headgear to protect the participant's hearing from noise having a potentially dangerous sound pressure level. Examples in the professional setting include, without limitation, construction workers, craftsman, airport ground support, pilots and military personnel. Sportsmen who often encounter loud noises include hunters and any individuals who recreationally use firearms. These individuals must don earplugs, earmuffs, or similar headgear in order to prevent hearing loss or injury to the user's hearing.

In addition, these users often opt to use some form of protective eyewear in conjunction with headgear. For example, there is often a risk of eye damage in the event of eye contact with an errantly ejected shell from a firearm. Likewise, there is often a risk of eye contact with debris when the user is engaged in a professional activity such as construction, woodworking, or use of heavy machinery.

U.S. Pat. Nos. 8,621,664 and 9,168,176 disclose eye protection systems in combination with headgear, where the eye wear is releasably connectable to the headgear by a magnetic connection. One issue with such systems is that many potential users have existing headgear and would prefer not to purchase new headgear. U.S. Pat. No. 9,168,176 discloses the possibility of a kit having eyewear and connectors, wherein connectors can be attached to existing headgear by an adhesive. However, such a system is difficult for an end user to properly utilize and has otherwise proven impractical.

Accordingly, it would be desirable to provide an eyewear attachment assembly that better facilitates end user attachment to existing headgear.

### SUMMARY OF THE INVENTION

In one aspect, an eyewear attachment assembly is provided for connecting eyewear to headgear. The eyewear attachment assembly includes a lens unit configured for protecting a user's eyes, the lens unit including at least one lens and a frame, the lens unit including side located first attachment units extending rearwardly from the frame. A pair of second attachment units are configured for connection to headgear, wherein each second attachment unit comprises a forward side and a rearward side, wherein, for each second attachment unit, the rearward side is configured for snap-connection onto a headgear feature such that the second attachment unit can be selectively removed from the headgear feature by pulling the second attachment unit away from the headgear feature. Each first attachment unit includes a first magnetic connector and the forward side of each second attachment unit includes a second magnetic connector, wherein the first magnetic connectors are magnetically attracted to the second magnetic connectors for holding the lens unit to the headgear while enabling removal of the lens unit by pulling the first magnetic connectors away from the second magnetic connectors. A removal force required to pull the first magnetic connectors away from the second magnetic connectors is less than a removal force required to pull the second attachment units off of the headgear features.

In another aspect, a method of adapting headgear for receiving eyewear, the headgear including a first earmuff with a first pivot bracket facing forwardly and a second earmuff with a second pivot bracket facing forwardly involves: utilizing a first attachment bracket with a snap-connection feature and a second attachment bracket with a snap-connection feature; snap-connecting the first attachment bracket onto the first pivot bracket such that the first attachment bracket will pivot with the first pivot bracket; and snap-connecting the second attachment bracket onto the second pivot bracket such that the second attachment bracket will pivot with the second pivot bracket.

In another aspect, a method of adapting headgear for receiving eyewear is provided, the headgear including a first earmuff with a first pivot bracket facing forwardly and a second earmuff with a second pivot bracket facing forwardly, wherein the first pivot bracket lacks any magnet and the second pivot bracket lacks any magnet. The method involves: removing the first pivot bracket from the first earmuff; removing the second pivot bracket from the second earmuff; snap-connecting a first attachment bracket to the first earmuff, the first attachment bracket carrying a first magnet; snap-connecting a second attachment bracket to the second earmuff, the second attachment bracket carrying a second magnet.

In a further aspect, an eyewear attachment assembly is provided for configuring headgear to magnetically retain eyewear. The eyewear attachment assembly includes first and second attachment brackets, wherein a rearward side of each of the first and second attachment brackets includes a concave region configured for snap-connection onto a respective headgear feature, such as a pivot pin or a pivot bracket pivotably mounted on the pivot pin.

In yet another aspect, an eyewear attachment assembly is provided for configuring headgear to magnetically retain eyewear. The eyewear attachment assembly includes first and second attachment brackets, wherein a rearward side of each of the first and second attachment brackets is configured for snap-connection onto a respective headgear feature.

The details of one or more embodiments are set forth in the accompanying drawings and the description below. Other features, objects, and advantages will be apparent from the description and drawings, and from the claims.

### BRIEF DESCRIPTION OF THE DRAWINGS

FIGS. 1-5 show a first embodiment of an eyewear attachment assembly;

FIGS. 6-10 show another embodiment of an attachment unit for an eyewear attachment assembly;

FIGS. 11-15 show another embodiment of an attachment unit for an eyewear attachment assembly;

FIGS. 16-20 show another embodiment of an attachment unit for an eyewear attachment assembly;

FIGS. 21-25 show another embodiment of an attachment unit for an eyewear attachment assembly;

FIGS. 26-30 show another embodiment of an attachment unit for an eyewear attachment assembly;

FIGS. 31-34 show an embodiment of an attachment unit for an eyewear attachment assembly, where the attachment unit is designed to replace the pivot bracket on the headgear;

FIGS. 35-40 show another embodiment of an attachment unit for an eyewear attachment assembly, where the attachment unit is designed to replace the pivot bracket on the headgear; and

FIGS. 41-44 show another embodiment of an attachment unit for snap-connecting on the pivot bracket of headgear.

### DETAILED DESCRIPTION

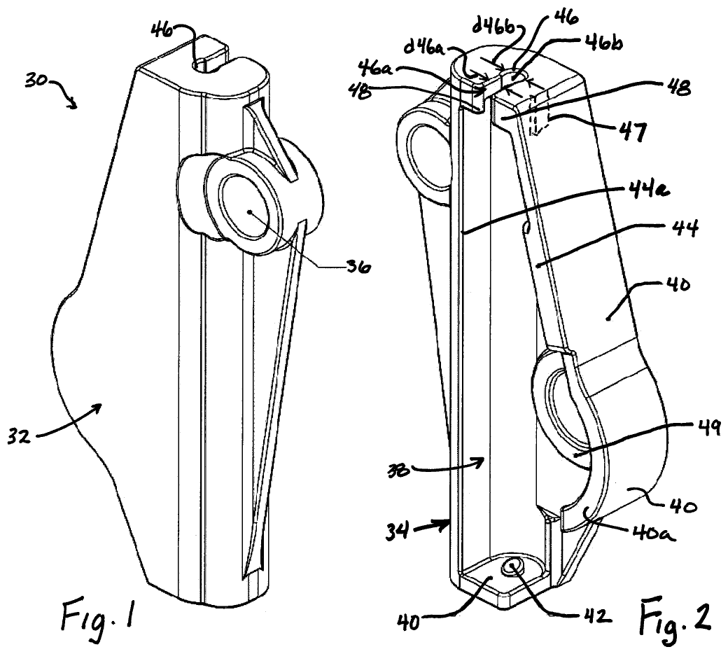

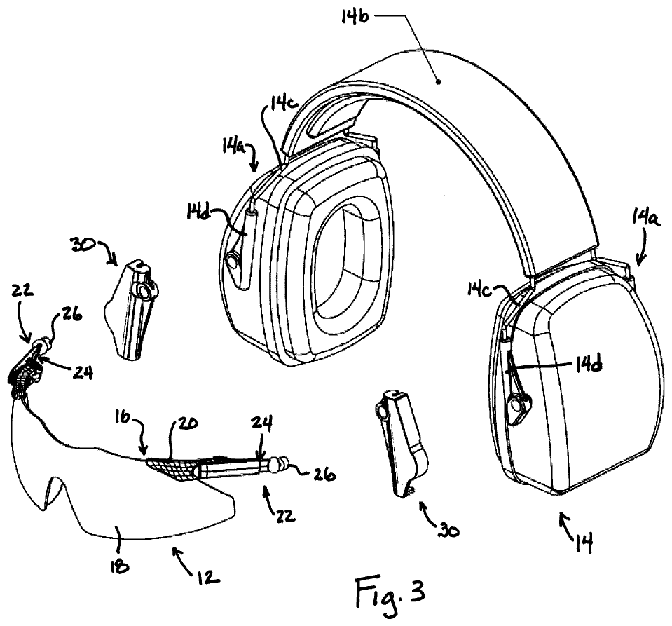

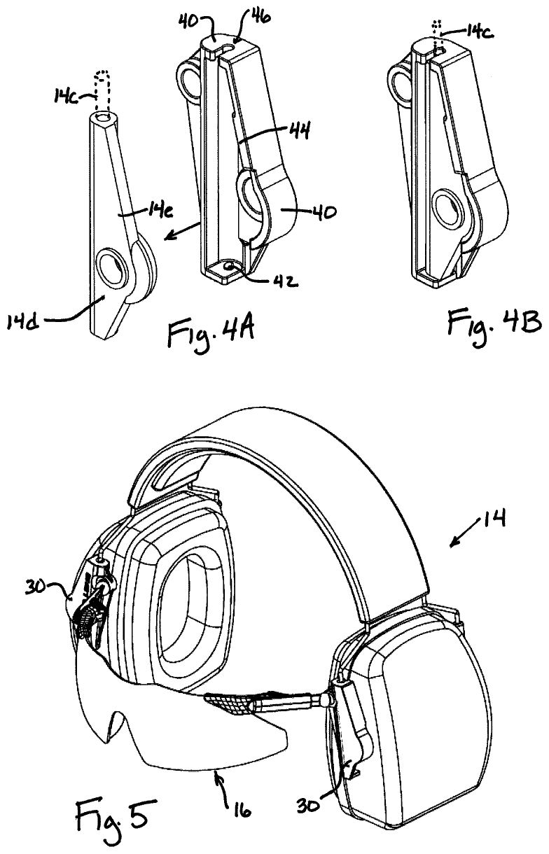

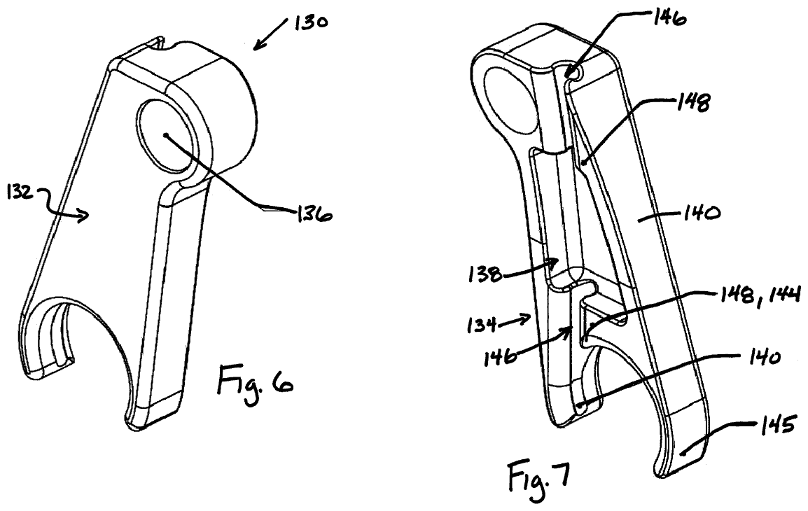

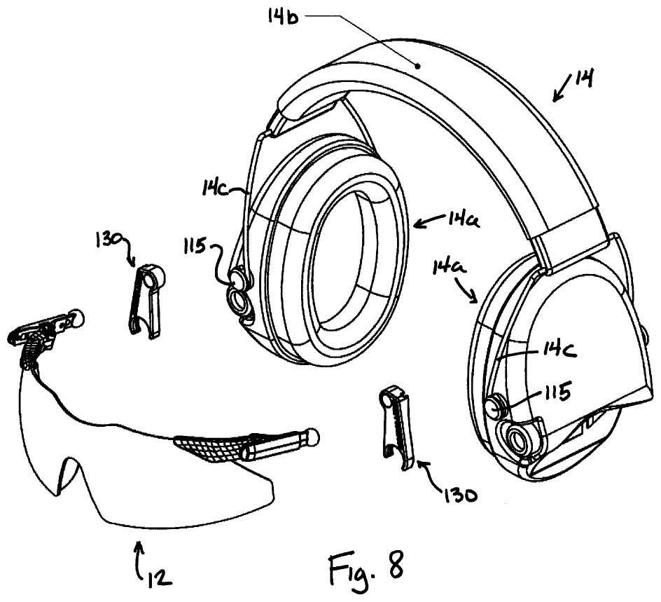

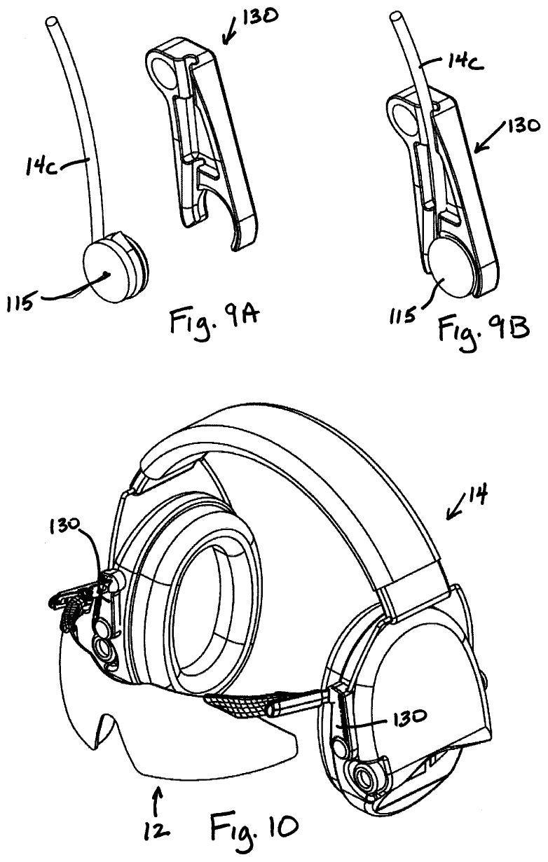

Referring to FIGS. 1-5, an eyewear attachment assembly for connecting eyewear 12 to headgear 14 is provided. Here, the eyewear takes the form of a lens unit 16 configured for protecting a user's eyes, where the lens unit 16 includes at least one lens 18 and a frame 20. The lens unit 16 also includes side located attachment units 22 extending rearwardly from the frame. The side located attachment units may, consistent with embodiments described in U.S. Pat. Nos. 8,621,664 and 9,168,176, be formed by telescoping members 24 having distal ends with an associated magnetic connector 26 (e.g., a magnet or magnetic material).

Hear, the headgear 14 includes earmuffs 14a and a connecting band 14b that interconnects the earmuffs vis engagement with a yoke 14c, where the yoke 14c engages pivot brackets 14d that are pivotable relative to the earmuffs 14a.

A pair of attachment units 30 are configured for connection to the headgear 14. The attachment brackets 30 will, typically, have a same configuration, except that one bracket will be a mirror image of the other. Each attachment unit 30 (e.g., a bracket) includes a forward side 32 and a rearward side 34, and the rearward side 34 is configured for snap-connection onto a headgear feature. This snap-connection also allows the attachment units 30 to be removed from the headgear by pulling the second attachment unit away from the headgear feature (e.g., overcoming the retention force of the snap-connection). Each attachment unit 30 includes a magnetic connector 36 (e.g., magnet or magnetic material) at the forward side 32. The magnetic connectors 26 are magnetically attracted to the magnetic connectors 36 for releasably holding the lens unit 16 to the headgear while enabling removal of the lens unit by pulling the magnetic connectors 26 away from the magnetic connectors 36 (e.g., overcoming the magnetic retention force). In embodiments, a removal force required to pull the magnetic connectors 26 away from the magnetic connectors 36 is less than a removal force required to pull the attachment units 30 off of the headgear features.

Here, the rearward side 34 of each attachment unit 30 is configured for snap-connection onto at least one of the pivot brackets 14d of the headgear or the yoke 14c. In many embodiments, the attachment units are configured for snap-connection to both the pivot bracket and the yoke. In embodiments, once connected, the attachment units 30 will pivot with the pivot brackets 14d.

Here, the rearward side 34 of each attachment unit 30 has one or more concave regions or cavities 38, with at least one retaining feature projecting into or alongside the concave region. In embodiments, the concave region is 38 bounded at least in part by an edge wall or walls 40, and the at least one retaining feature includes one or both of (i) a retaining projection 42 (e.g., a pin, which may be tapered in an orientation facing the open side of the concave region to facilitate pressing onto the headgear bracket) projecting inwardly from the lower part of the edge wall 40 and spaced forwardly of a rear or distal end 40a of the edge wall and/or (ii) a retaining clip 44 or clips (e.g., in the form of ledge(s), lips or other extension(s) that overhang part of the concave region or cavity, which also may be tapered) extending from the edge wall. When attached to the headgear, the projection 42 will snap into an opening at the bottom of the pivot bracket 14d, and the retaining clip 44 will seat behind an edge part 14e of the pivot bracket, thereby providing the desired connection and retention. Here, the long wall portion defining the cavity also includes a retaining the clip 44a in the form of a modest ledge or lip that overhangs part of the cavity. In addition, the edge wall 40 has an opening 46 for receiving the headgear yoke 14c. Here, the opening 46 is a slot opening such that the edge wall 40 includes a rearwardly extending entry slot part 46a through which the headgear yoke can enter into the opening. The entry slot part 46a is shaped as an entry throat having a portion with a dimension d46a that is smaller than a dimension d46b of the main part 46b of the opening such that the headgear yoke snap-fits into the main part 46b of the opening by moving through the entry throat. A pair of downwardly extending clips 48 (e.g., extending ledges) are provided at both sides of the slot to snap behind portions of the headgear pivot bracket 14b. Here, the concave region may also include a rearwardly extending retaining lip 47 that engages part of the headgear to prevent rotation of the attachment unit relative to the pivot bracket, and a rearwardly projecting ring part 49 that seats against an outwardly facing part of the pivot bracket 14d.

Various configurations of protective headgear are available in the market, each typically including its own variation of a pivot bracket and yoke, with differing configurations. In the below embodiments, like numbers refer to like parts (e.g., parts having similar functions) and, when considering the three digit reference numbers below, the last two digits should be considered when determining like numbers.

FIGS. 6-10 show another embodiment in which the attachment units 130 include forward side 132, rearward side 134, magnetic connectors 136, concave region 138, edge walls 140, slot openings 146, and clip parts 144, 148. The attachments units 130 snap-connection over pivot pins 115 of the headgear 14, which also includes earmuffs 14a, a connecting headband 14b and yokes 14c. Here, the pivot pins 115 are fixed to the earmuffs, and wires bent in circular loops are attached to the pivot pins allowing the wires to rotate around the pins. The pivot pins have an enlarged distal end or head to retain the wires axially on the pins.

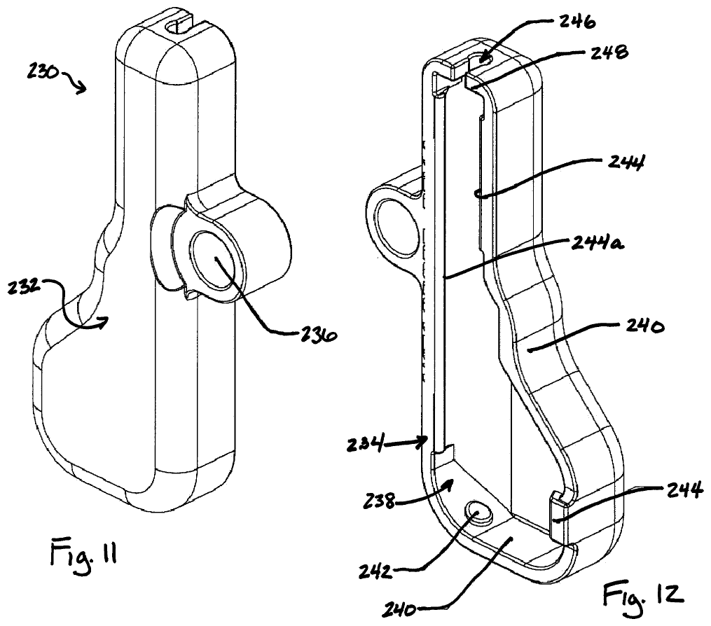

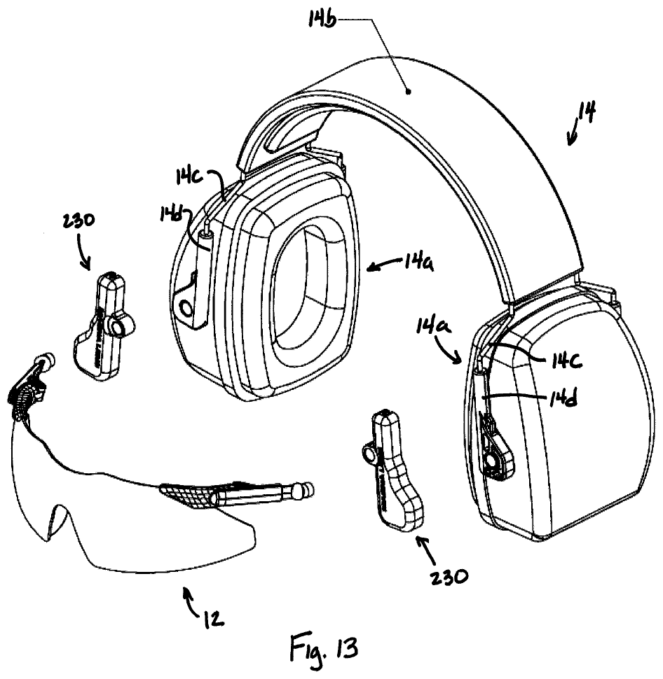

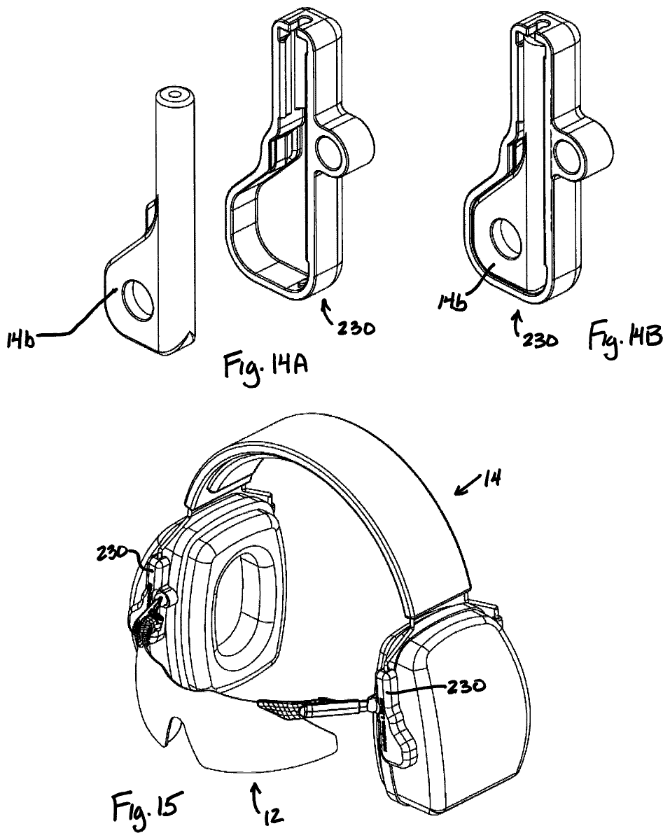

FIGS. 11-15 show another embodiment in which the attachment units 230 include forward side 232, rearward side 234, magnetic connectors 236, concave regions 238, edge walls 240, pins 242, slot openings 246, and clip parts 244, 244a, 248. The attachments units 230 snap-connect over pivot brackets 14d of the headgear 14, which also includes earmuffs 14a, a connecting headband 14b and yokes 14c.

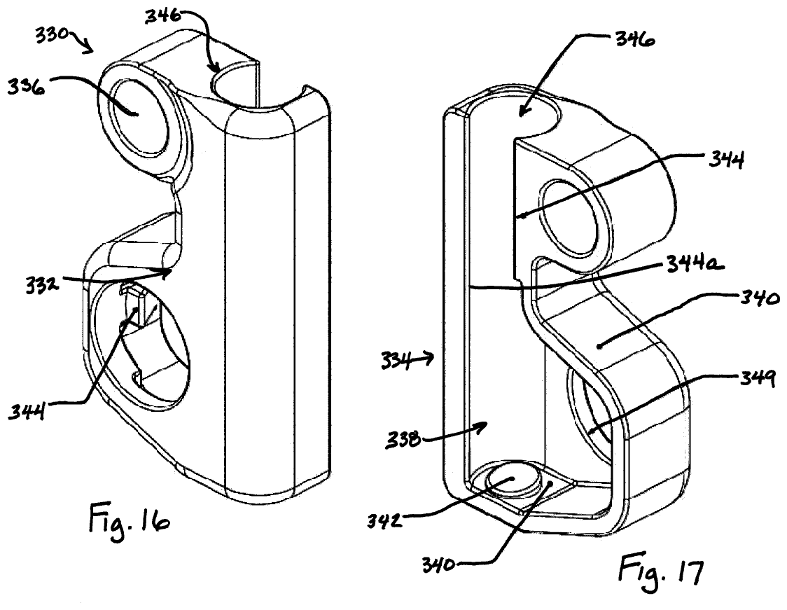

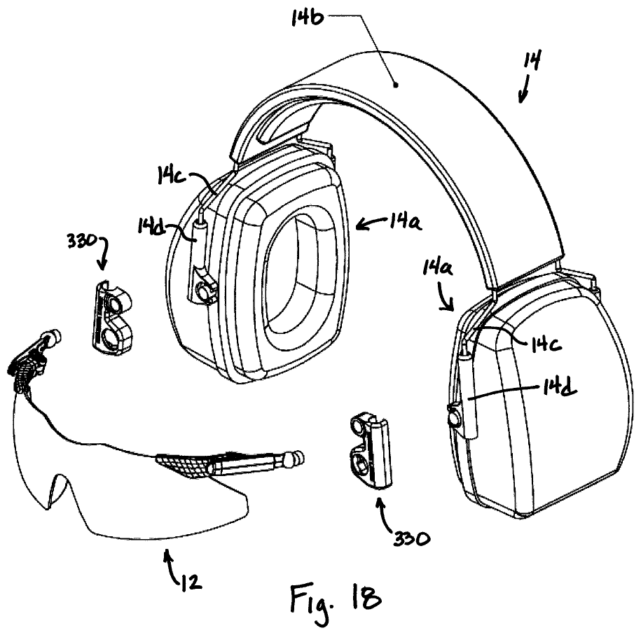

FIGS. 16-20 show another embodiment in which the attachment units 330 include forward side 332, rearward side 334, magnetic connectors 336, concave regions 338, edge walls 340, pins 342, slot openings 346, clip parts 344, 344a and ring part 349. The attachments units 330 snap-connect over pivot brackets 14d of the headgear 14, which also includes earmuffs 14a, a connecting headband 14b and yokes 14c.

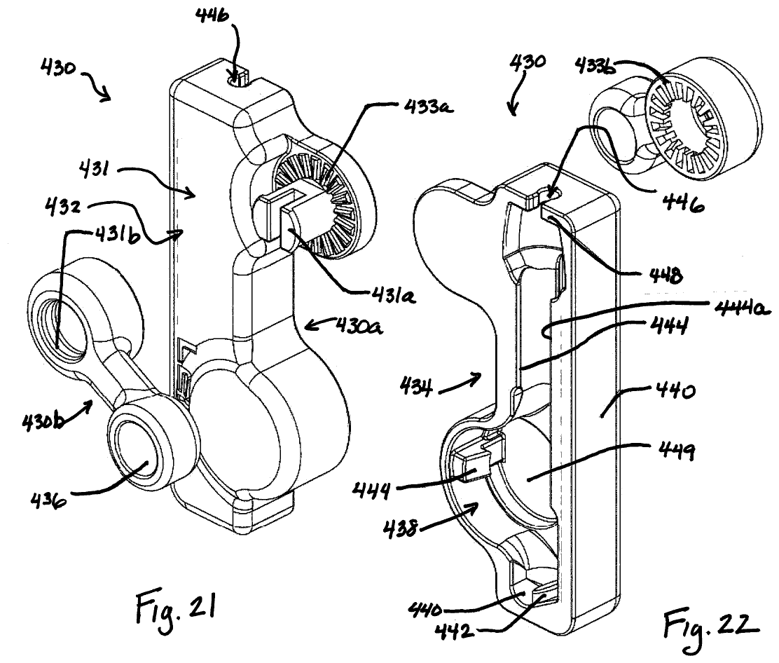

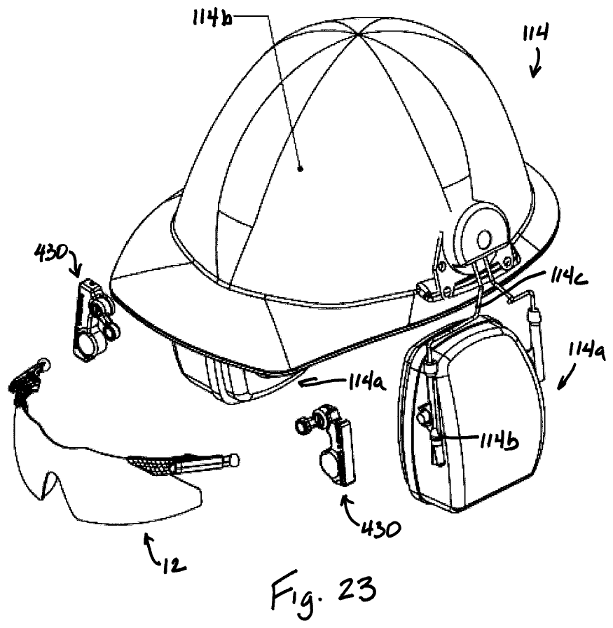

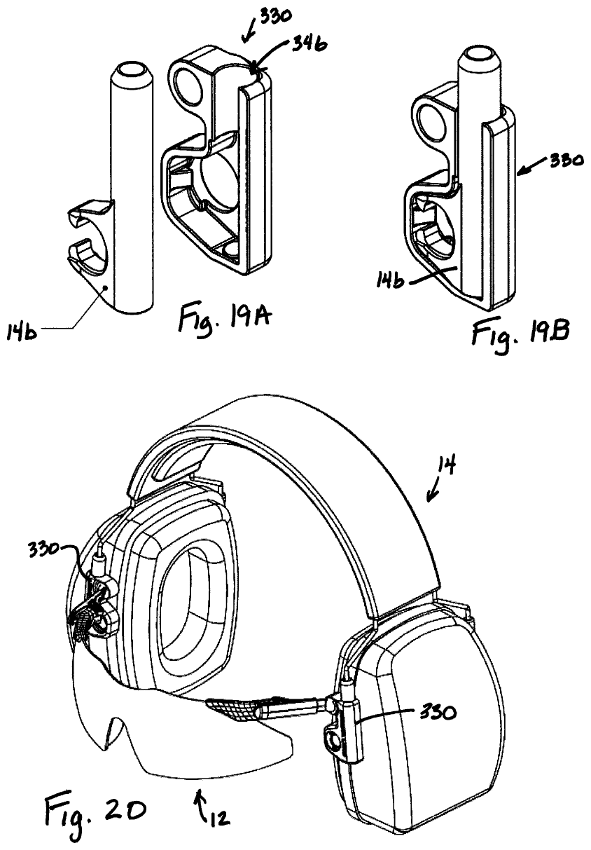

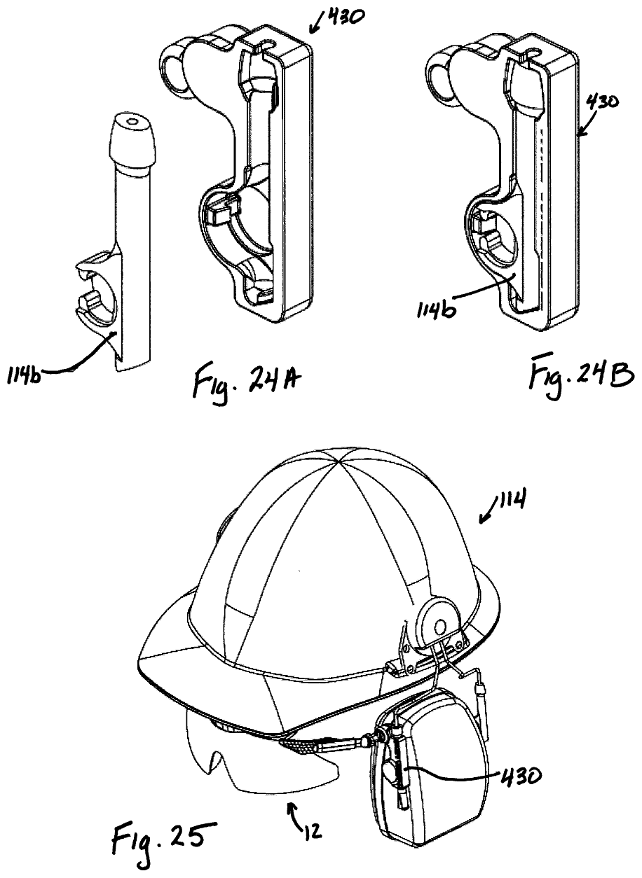

FIGS. 21-25 show a further embodiment of in which the attachment units 430 include two parts 430a, 430b that are connected together at a snap together pivot connection 431 that allows the two parts to pivot relative to each other utilizing a pivot post 431a that engages with an opening 431b. Mating detent features 433a, 433b are provided to stabilize the rotational position of part 430b relative to part 430, Part 430a is configured for snap-connection onto the headgear 114, which in this case includes earmuffs 114a, a helmet 114b, yokes 114c and pivot brackets 114d. Part 430a includes forward side 432, rearward side 434, concave regions 438, edge walls 440, pins 442, slot openings 446, clip parts 444, 444a, 448 and ring part 449. Part 430b carries the magnet 436. The attachments units 430 snap-connect over pivot brackets 114d of the headgear 114.

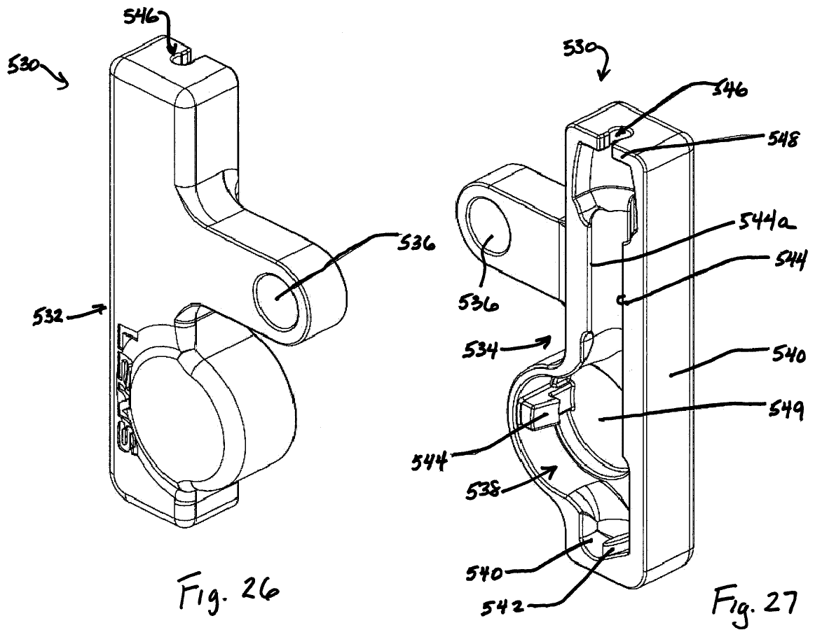

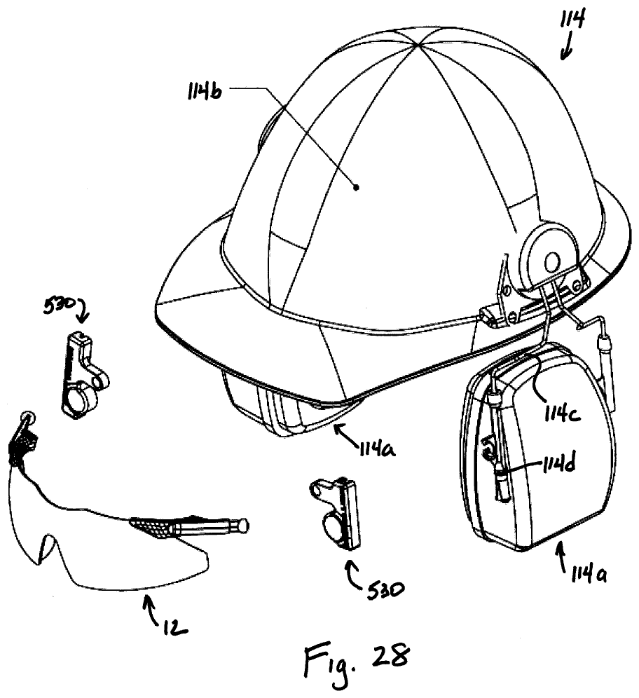

FIGS. 26-30 show a further embodiment of in which the attachment units 530 forward sides 532, rearward sides 434, magnetic connector 536, concave regions 538, edge walls 540, pins 542, slot openings 546, clip parts 544, 544a, 548 and ring part 549. The attachments units 530 snap-connect over pivot brackets 114d of the headgear 114, which also includes earmuffs 114a, helmet 114b and yokes 114c.

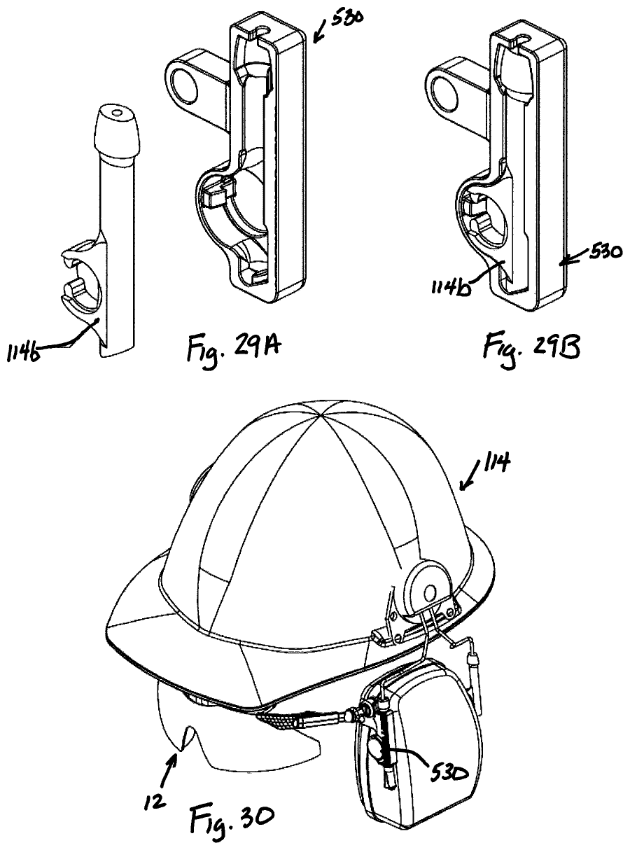

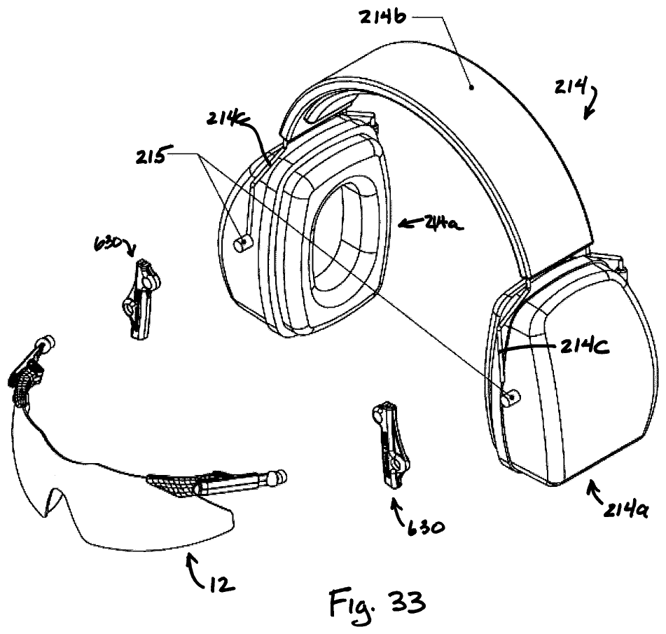

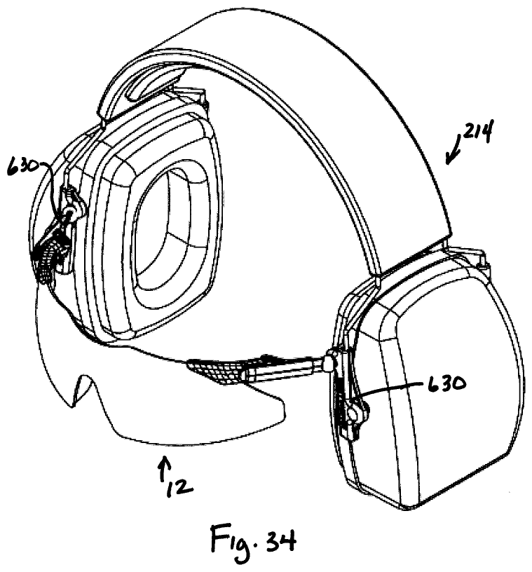

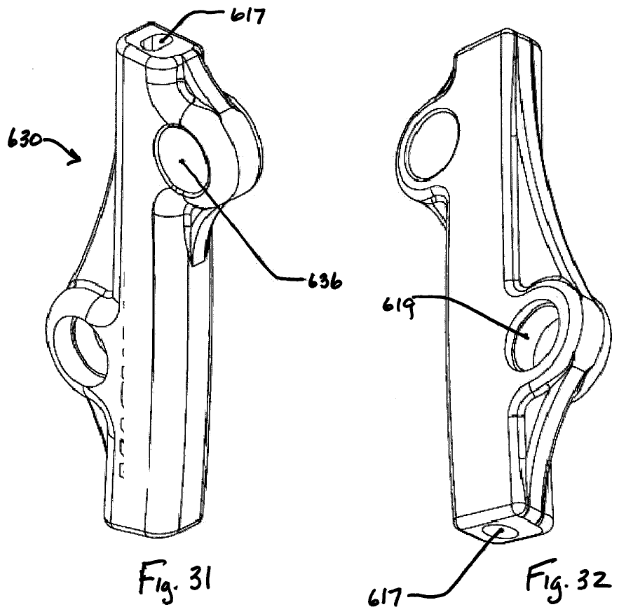

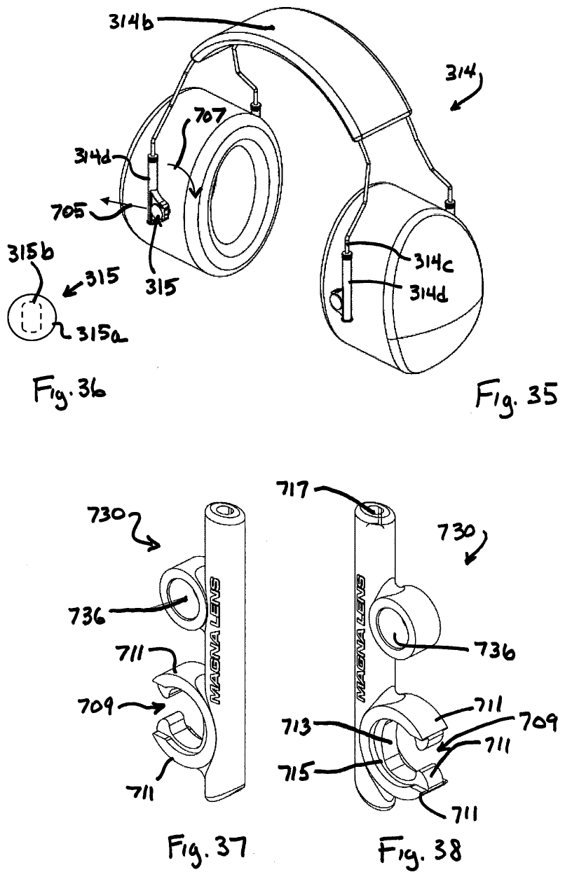

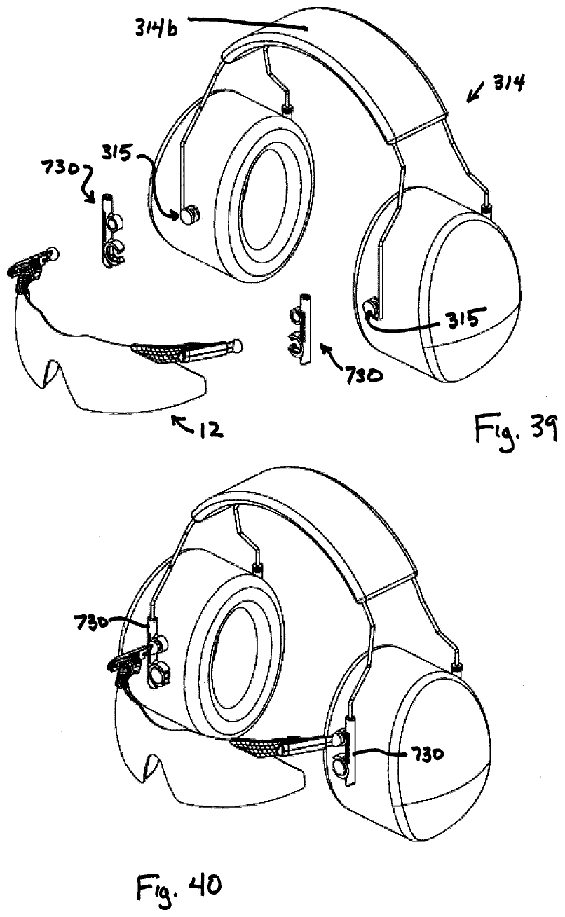

FIGS. 31-34 show an embodiment in which the attachment units 630 are configured to replace the pivot brackets of the headgear 214. Specifically, the pivot brackets of the headgear 214 are removed, and the attachment units 630 pivotably engage onto pivot posts 215 of the head gear 214 via openings 617 and also engage with the head gear yokes 214c (the yokes slidingly engage into openings 617 in the bracket and may be retained by, for example, an internal feature of the openings 617). The connection of the attachment units 630 to the headgear may be a snap-connection onto the headgear pivot posts 215. The head gear 214 also includes headband 214b.

FIGS. 35-40 show another embodiment in which the attachment units 730 are configured to replace the pivot brackets 314d of the headgear 314, which includes earmuffs 314a, headband 314b and yokes 314c. Hear, the pivot brackets 314d are pivotably mounted on pivot posts 315 that have an enlarged distal head region 315a at the end of a post shaft 315b. The pivot brackets have gripping clips in the form of arms that form a C-shape to engage the post shaft 315b inward of the head region 315a. The post shaft 315b is out-of-round, and cooperates with the arms, such that removal of the pivot brackets in one orientation of the earmuffs 314a is not possible (e.g., with the earmuffs oriented as shown, the larger dimension of the post shaft 315b prevents the pivot bracket 314d from being removed in a direction 705). On the other hand, if the earmuff 314a, along with the post shaft 315b, is rotated by 90 degrees relative to the pivot bracket (per arrow 707), then the smaller dimension of the post shaft 315b will align with the slot of the C-shape, allowing the pivot bracket 314d to release off of the post shaft 315 when sufficient force is applied.

The attachment units 730 pivotably engage onto pivot posts 315 of the head gear 314 in a similar manner, vis the slot 709 between the arms 711. The arms 711 include a ledge feature 713 that defines a recessed surface 715 that will seat against the backside of the head region 315a. The attachment units 730 carry magnets 736, and also include an end opening 717 to engage with the head gear yokes 314c (the yokes slidingly engage into openings 717 in the bracket and may be retained by, for example, an internal feature of the openings 717). The connection of the attachment units 730 to the headgear is a snap-connection onto the headgear pivot posts 315.

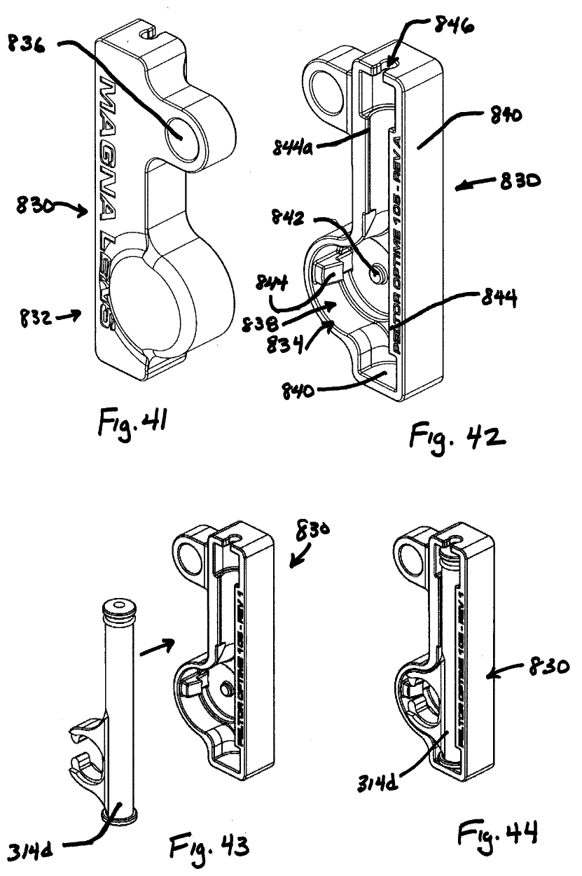

FIGS. 41-44 shows a snap-connectable attachment unit 830 configured for attachment to the pivot brackets 314d of FIG. 35, without requiring removal of the pivot brackets 314d. Attachment unit 830 includes forward side 832, rearward side 834, magnet 836, concave region 838, edge walls 840, pin 842, slot opening 846 and clip parts 844, 844a.

All of the foregoing embodiments snap-connect to the headgear (e.g., to the pivot bracket and/or the yoke of the headgear or to the pivot post of the headgear). Generally the snap-connection may be along a direction aligned with the pivot axis of the pivot bracket or pivot post may be utilized. However, variations in which the attachment units snap-connect along a direction that is transvers to the pivot axis are also possible.

It is to be clearly understood that the above description is intended by way of illustration and example only, is not intended to be taken by way of limitation, and that other changes and modifications are possible.

1. An eyewear attachment assembly for connecting eyewear to headgear, the eyewear attachment assembly comprising:
a lens unit configured for protecting a user's eyes, the lens unit including at least one lens and a frame, the lens unit including side located first attachment units extending rearwardly from the frame;
a pair of second attachment units configured for connection to headgear, wherein each second attachment unit comprises a forward side and a rearward side, wherein, for each second attachment unit, the rearward side is configured for snap-connection onto a headgear feature such that the second attachment unit can be selectively removed from the headgear feature by pulling the second attachment unit away from the headgear feature;
wherein each first attachment unit includes a first magnetic connector and the forward side of each second attachment unit includes a second magnetic connector, wherein the first magnetic connectors are magnetically attracted to the second magnetic connectors for holding the lens unit to the headgear while enabling removal of the lens unit by pulling the first magnetic connectors away from the second magnetic connectors;
wherein a removal force required to pull the first magnetic connectors away from the second magnetic connectors is less than a removal force required to pull the second attachment units off of the headgear features.

2. The eyewear attachment assembly of claim 1, wherein the rearward side of each second attachment unit is configured for snap-connection onto at least one of a pivot bracket of the headgear or a yoke extending from the pivot bracket.

3. The eyewear attachment assembly of claim 1, wherein the rearward side of each second attachment unit is configured for snap-connection to both a pivot bracket of the headgear and a yoke extending from the pivot bracket.

4. The eyewear attachment assembly of claim 1, wherein the rearward side of each second attachment unit has at least one concave region, wherein at least one retaining feature projects into or alongside the concave region.

5. The eyewear attachment assembly of claim 4, wherein the concave region is bounded in part by an edge wall, wherein the at least one retaining feature includes at least one of (i) a retaining pin projecting inwardly from the edge wall and spaced forwardly of a distal end of the edge wall or (ii) a retaining clip extending from the edge wall.

6. The eyewear attachment assembly of claim 1, wherein the rearward side of each second attachment unit has at least one concave region, wherein the concave region is bounded in part by an edge wall having an opening for receiving a headgear yoke.

7. The eyewear attachment assembly of claim 6, wherein the edge wall includes a rearwardly extending entry slot through which a headgear yoke can enter into the opening, wherein the entry slot is shaped as an entry throat having a portion with a dimension smaller than a dimension of the opening such that a headgear yoke snap-fits into the opening by moving through the entry throat.

8. The eyewear attachment assembly of claim 1, wherein each first magnetic connector comprises a magnet and each second magnetic connector comprises a magnet or a magnetic material.

9. The eyewear attachment assembly of claim 1, wherein the headgear feature is a pivot post.

10. A method of adapting headgear for receiving eyewear, the headgear including a first earmuff with a first pivot feature facing forwardly and a second earmuff with a second pivot feature facing forwardly, the method comprising:
utilizing a first attachment bracket with a snap-connection feature and a second attachment bracket with a snap-connection feature;
snap-connecting the first attachment bracket onto the first pivot feature such that the first attachment bracket will pivot with or about the first pivot feature;
snap-connecting the second attachment bracket onto the second pivot feature such that the second attachment bracket will pivot with or about the second pivot feature.

11. The method of claim 10, wherein the first attachment bracket carries a first magnetic connector that faces forwardly after connection of the first attachment bracket to the first pivot bracket, wherein the second attachment bracket carries a second magnetic connector that faces forwardly after connection of the second attachment bracket to the second pivot bracket.

12. The method of claim 11, wherein the first magnetic connector is a magnet and the second magnetic connector is a magnet.

13. The method of claim 11, further comprising the step of releasably attaching a lens unit to the headgear via magnetic attraction to the first and second magnetic connectors.

14. The method of claim 11 wherein the first pivot feature is one of a first pivot post or a first pivot bracket, wherein the second feature is a second pivot post or second pivot bracket.

15. A method of adapting headgear for receiving eyewear, the headgear including a first earmuff with a first pivot bracket facing forwardly and a second earmuff with a second pivot bracket facing forwardly, wherein the first pivot bracket lacks any magnet and the second pivot bracket lacks any magnet, the method comprising:
removing the first pivot bracket from the first earmuff;
removing the second pivot bracket from the second earmuff;
snap-connecting a first attachment bracket to the first earmuff, the first attachment bracket carrying a first magnet;
snap-connecting a second attachment bracket to the second earmuff, the second attachment bracket carrying a second magnet.

16. An eyewear attachment assembly for configuring headgear to magnetically retain eyewear, the eyewear attachment assembly comprising:
first and second attachment brackets, wherein a rearward side of each of the first and second attachment brackets is configured for snap-connection onto a respective headgear feature.

17. The eyewear attachment assembly of claim 16, wherein each of the first and second attachment brackets includes a concave region configured for snap-connection onto a respective headgear pivot bracket.

18. The eyewear attachment assembly of claim 17, wherein, for each of the first and second attachment brackets, at least one retaining feature projects into or alongside the concave region.

19. The eyewear attachment assembly of claim 18, wherein the concave region is bounded in part by an edge wall, wherein the at least one retaining feature includes at least one of (i) a retaining pin projecting inwardly from the edge wall and spaced forwardly of a distal end of the edge wall or (ii) a retaining clip extending from the edge wall.

20. The eyewear attachment assembly of claim 16, wherein the rearward side of each of the first and second attachment brackets is further configured with a slot for connection to a yoke extending from a headgear pivot bracket, wherein, for each of the first and second attachment brackets, the slot includes a main slot portion and an entry slot portion through which a headgear yoke can enter into the main slot portion, wherein the entry slot portion is shaped as an entry throat having a portion with a dimension smaller than a dimension of the main slot portion such that a headgear yoke snap-fits into the main slot portion by moving through the entry throat.
